(19) United States
(12) Patent Application Publication
(10) Pub. No.: US 2010/0274162 A1
(43) Pub. Date: Oct. 28, 2010

(54) Thermal Massager

(76) Inventor: Dale Marlyn Marie Evans, Orlando, FL (US)

Correspondence Address: GEORGE R. REARDON, 3356 STATION COURT, LAWRENCVILLE, GA, 30044, US

(21) Appl. No.: 12/427,831

(22) Filed: Apr. 22, 2009

(51) Int. Cl. A61F 7/00; A61H 1/00

(52) U.S. Cl. 601/15; 601/46

(57) ABSTRACT

The technology described herein provides a handheld massager. The handheld massager includes a housing dimensioned for handheld grip by a user; a massage head capsule within which a massage roller ball rotates; a thermoelectric assembly configured to selectively and incrementally heat and cool the massage roller ball; a temperature control to control a temperature of the thermoelectric device; a fluid reservoir configured to hold a massage fluid for dispensing to the massage roller ball; a fluid dispenser configured to control the release and volume of the massage fluid from the fluid reservoir; a vibrator motor configured to provide a vibration to the massager; a power source configured to provide power to the massager; user controls configured to variably control vibration, temperature, and power; and a controller configured to electronically couple and control the plurality of user controls, the power source, the fluid dispenser, the temperature control, and the thermoelectric assembly.

### FIELD OF THE INVENTION

The technology described herein relates generally to massage devices. More specifically, this technology relates to a handheld thermal massager utilizing heat and cold therapy, vibration, and passive dispensing of medicated, scented, and the like, massage fluid via an interchangeable rolling ball.

### BACKGROUND OF THE INVENTION

Massage is the beneficial practice of tissue manipulation. By way of example, massage can provide therapeutic advantages, alleviate pain, stimulate circulation, relieve tension, provide flexibility to muscles and joints, and improve range of motion, and so forth. Additionally, massage can provide psychological benefits such as lessening depression and anxiety and increasing sleep quality.

Related patents known in the art include the following: U.S. Pat. No. 1,947,042, issued to Glennan on Feb. 13, 1934, discloses a massage device. U.S. Pat. No. 4,116,233, issued to Scaduto on Sep. 26, 1978, discloses a portable massager. U.S. Published Patent application No. 2008/0154161 filed by Abbott and published on Jun. 26, 2008, discloses a handheld massager.

The foregoing patent and other information reflect the state of the art of which the inventors are aware and are tendered with a view toward discharging the inventors' acknowledged duty of candor in disclosing information that may be pertinent to the patentability of the technology described herein. It is respectfully stipulated, however, that the foregoing patent and other information do not teach or render obvious, singly or when considered in combination, the inventors' claimed invention.

### BRIEF SUMMARY OF THE INVENTION

In various exemplary embodiments, the technology described herein provides a handheld thermal massager utilizing heat and cold therapy, vibration, and passive dispensing of medicated, scented, and the like, massage fluid via an interchangeable rolling ball. The technology described herein can be utilized for personal use as well as for professional use, e.g., commercially.

In one exemplary embodiment, the technology described herein provides a handheld massager. The handheld massager includes a housing dimensioned for handheld grip by a user; a massage head capsule within which a massage roller ball rotates; a thermoelectric assembly configured to selectively and incrementally heat and cool the massage roller ball; a temperature control to control a temperature of the thermoelectric device; a fluid reservoir configured to hold a massage fluid for dispensing to the massage roller ball; a fluid dispenser configured to control the release and volume of the massage fluid from the fluid reservoir; a vibrator and rotator motor configured to provide a vibration and rotation to the massager; a power source configured to provide power to the massager; a plurality of user controls configured to variably control vibration, temperature, and power; and a controller configured to electronically couple control the plurality of user controls, the power source, the fluid dispenser, the temperature control, and the thermoelectric assembly.

The massage roller ball is configured to be heated and cooled selectively and incrementally to provide variable hot and cold therapy.

The massage head capsule is detachable from the massager housing and is both replaceable and interchangeable, thereby providing the massager with a plurality of massage options and a plurality of massage roller balls of varying sizes.

In at least one embodiment, the fluid dispenser is a cartridge that is both interchangeable and disposable.

In various embodiments, the thermoelectric assembly configured to selectively and incrementally heat and cool the massage roller ball further includes one or more of a fan, a heat sink, a universal heat exchange device, and a conversion heat exchange device. The massage roller ball is a metal ball in at least one embodiment.

In various embodiments, the power sources include one or more of rechargeable battery and charger, solar cells, whereby the massager can be operated cordlessly.

The temperature control to control a temperature of the thermoelectric device is selectively and incrementally controlled by the user operating one or more of the plurality of user controls to heat and cool the massage roller ball.

The plurality of user controls configured to variably control vibration, temperature, and power further comprises a vibration on/off switch and an intensity of vibration control.

The controller is further configured to automatically shut-off power to the massager once a predetermined time has elapsed and once a predetermined temperature has been exceeded.

The massager can also include a saturating sponge disposed within the massage head capsule and configured to absorb dispensed fluid and provide an even distribution of the fluid onto the massage roller ball as it rotates over the saturating sponge.

The massager can also include a quick release button disposed upon the housing of the massager and configured to be actuated by the user to remove the massage capsule from the massager for interchangeability and replaceability.

The massager can also include a removable protective cap configured to protect the massage capsule and massage roller ball from debris and damage. The protective cap can be threaded.

The massager can further include a temperature display window configured to provide the user with a current temperature of the massage roller ball.

The massager can further include a turning wheel having a stem disposed upon the massage capsule and configured for operative engagement to release massage fluid from the massage reservoir.

There has thus been outlined, rather broadly, the more important features of the technology in order that the detailed description thereof that follows may be better understood, and in order that the present contribution to the art may be better appreciated. There are additional features of the technology that will be described hereinafter and which will form the subject matter of the claims appended hereto. In this respect, before explaining at least one embodiment of the technology in detail, it is to be understood that the invention is not limited in its application to the details of construction and to the arrangements of the components set forth in the following description or illustrated in the drawings. The technology described herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the technology described herein.

Further objects and advantages of the technology described herein will be apparent from the following detailed description of a presently preferred embodiment which is illustrated schematically in the accompanying drawings.

### BRIEF DESCRIPTION OF THE DRAWINGS

The technology described herein is illustrated with reference to the various drawings, in which like reference numbers denote like device components and/or method steps, respectively, and in which:

FIG. 1 is a front perspective view of a handheld thermal massager, according to an embodiment of the technology described herein;

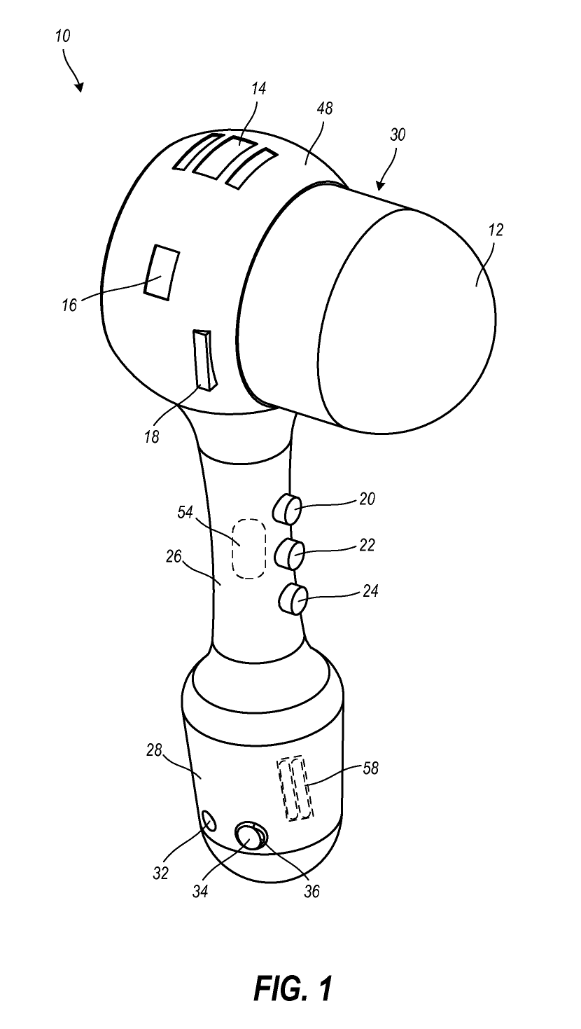

FIG. 2 is a rear perspective view of the handheld thermal massager handheld thermal massager depicted in FIG. 1;

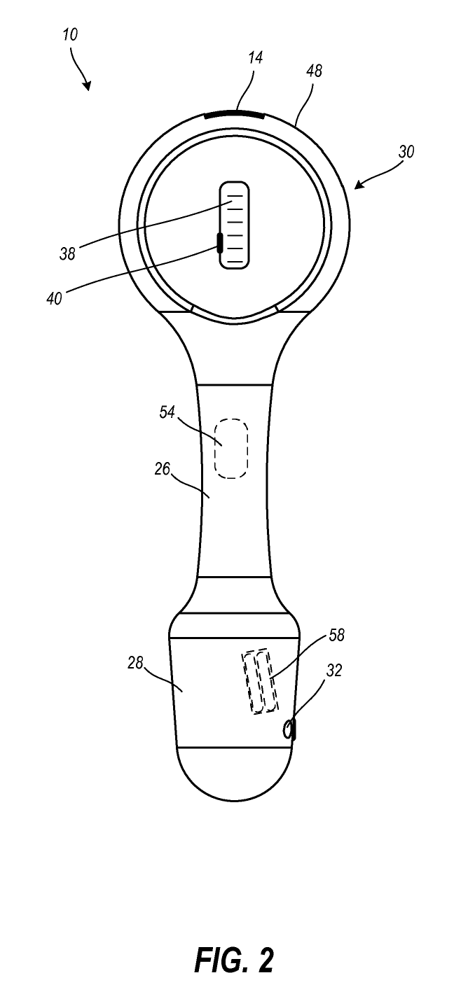

FIG. 3 is a side perspective view of the handheld thermal massager handheld thermal massager depicted in FIG. 1;

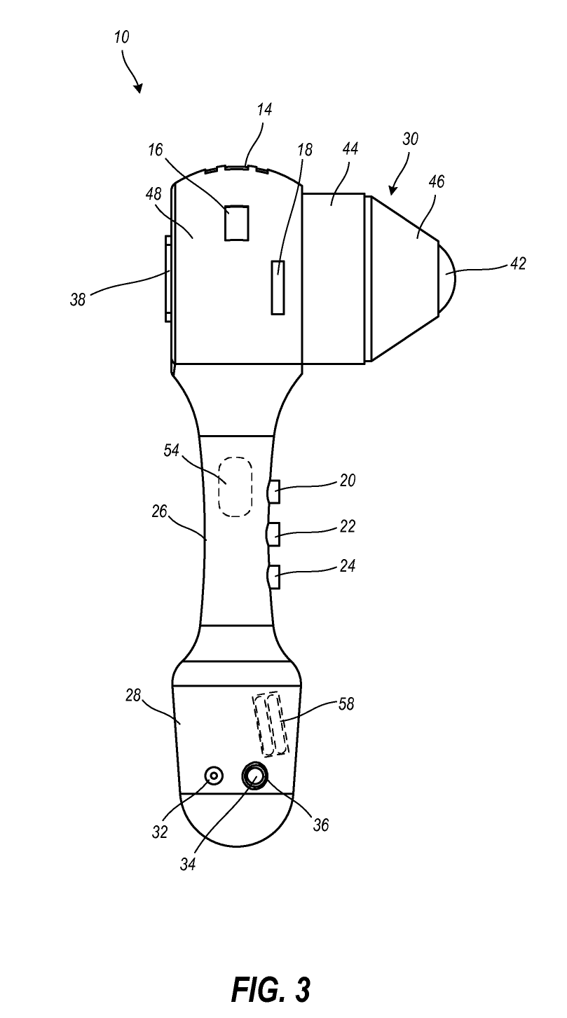

FIG. 4 is a top perspective view of the handheld thermal massager handheld thermal massager depicted in FIG. 1;

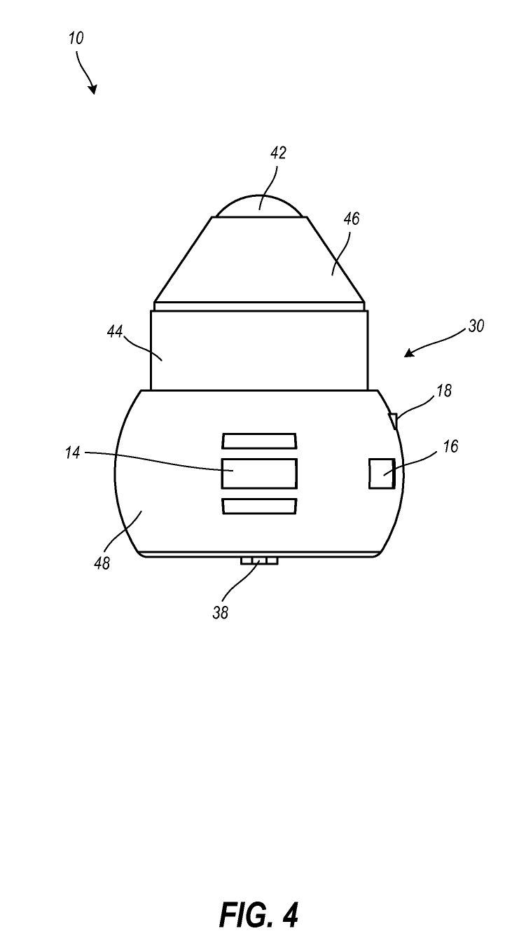

FIG. 5 is a front perspective view of the handheld thermal massager handheld thermal massager depicted in FIG. 1, with the cap removed, and illustrating, in particular, the head assembly;

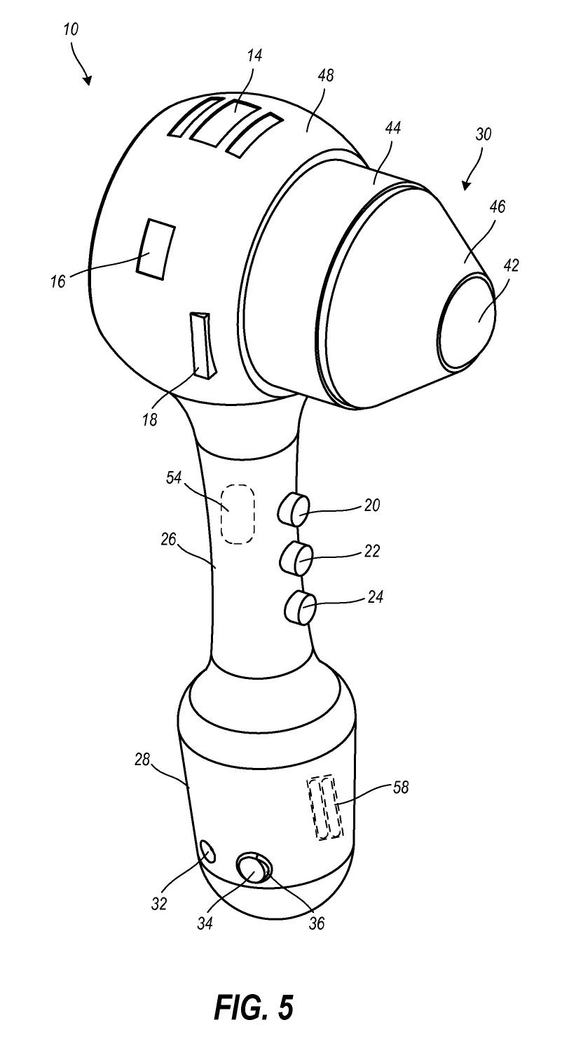

FIG. 6 is a front perspective view of the handheld thermal massager handheld thermal massager depicted in FIG. 1, with the cap removed, and illustrating, in particular, the detachability of the head assembly;

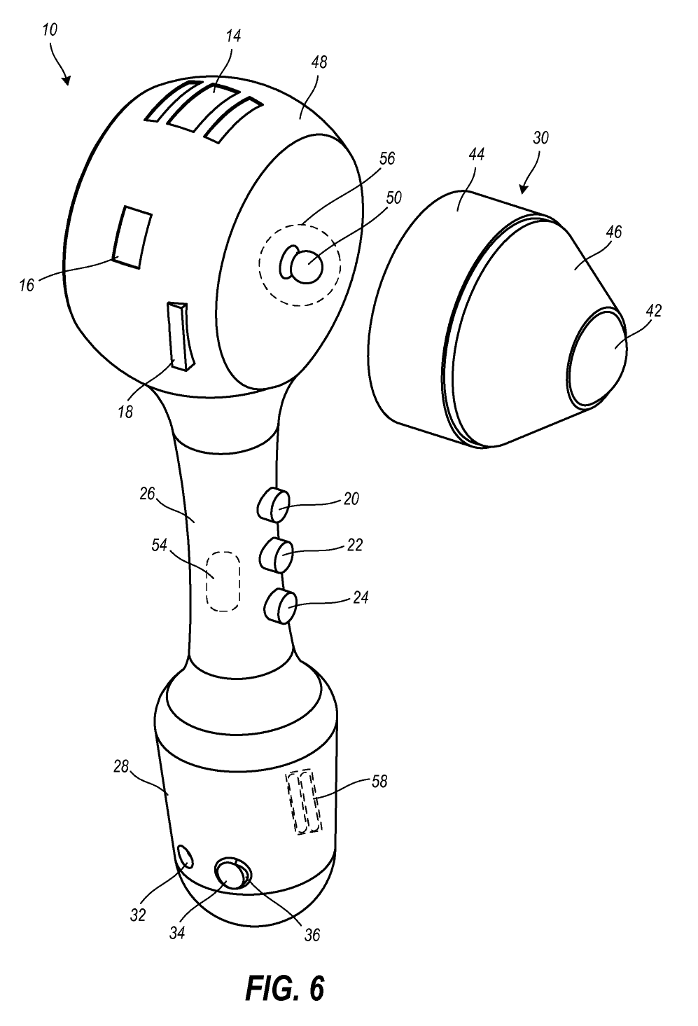

FIG. 7 is an expanded front perspective view of the handheld thermal massager handheld thermal massager depicted in FIG. 1 and the head assembly; and

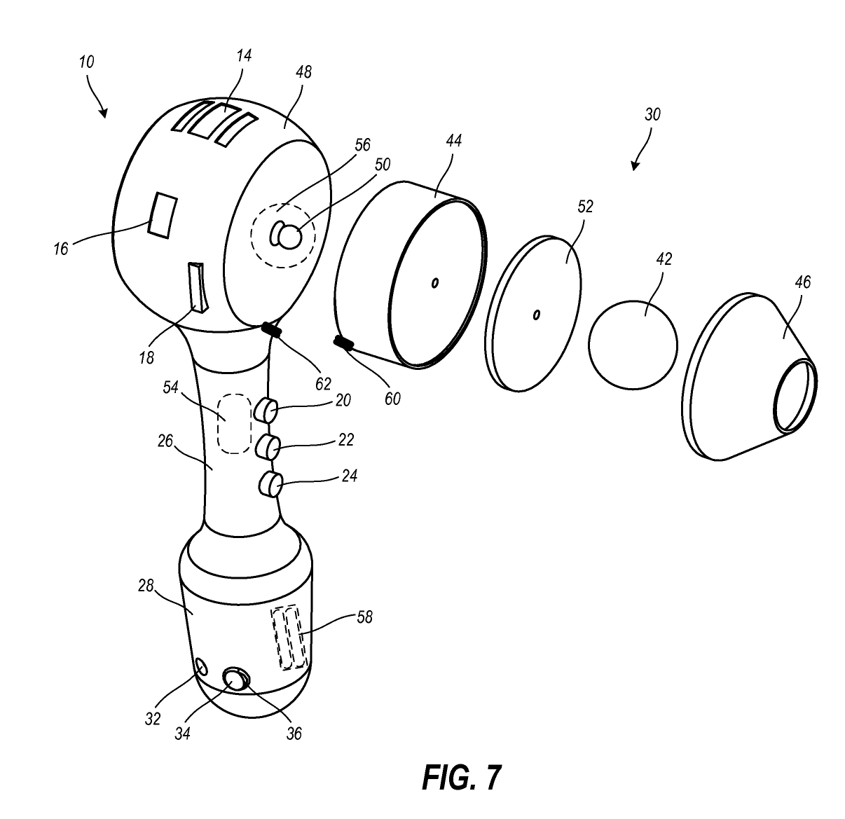

FIG. 8 is an expanded rear perspective view of the handheld thermal massager handheld thermal massager depicted in FIG. 1 and the head assembly.

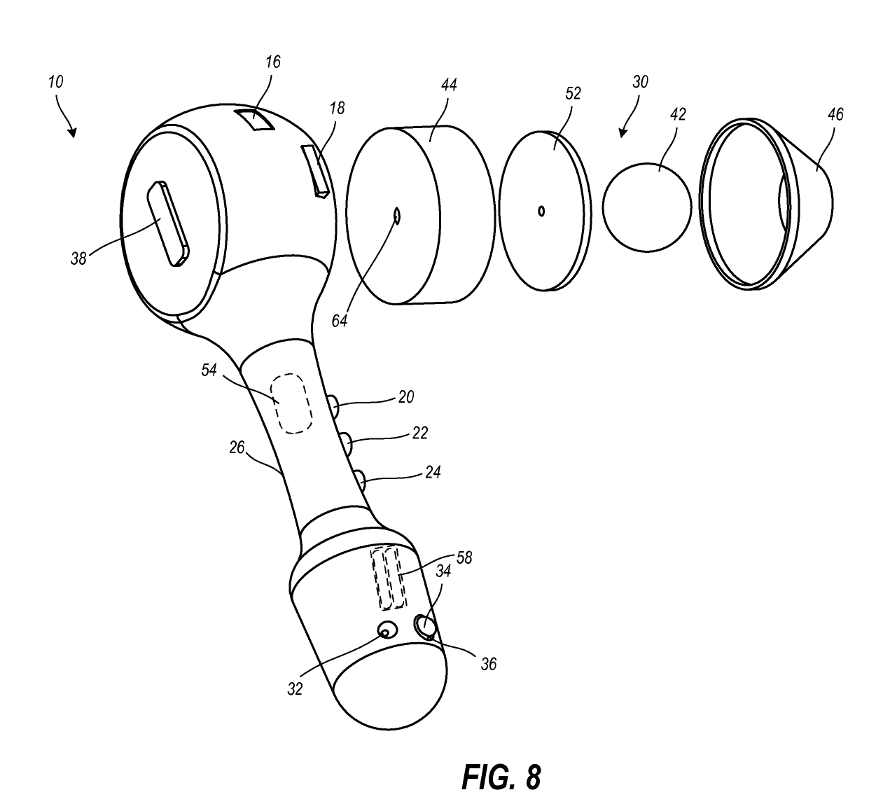

### DETAILED DESCRIPTION OF THE INVENTION

Before describing the disclosed embodiments of this technology in detail, it is to be understood that the technology is not limited in its application to the details of the particular arrangement shown here since the technology described is capable of other embodiments. Also, the terminology used herein is for the purpose of description and not of limitation.

In various exemplary embodiments, the technology described herein provides a handheld thermal massager utilizing heat and cold therapy, vibration, and passive dispensing of medicated, scented, and the like, massage fluid via an interchangeable rolling ball.

Referring now to the Figures, a massager 10 is shown. The massager 10 is configured to be handheld. The massager 10 includes a housing dimensioned for handheld grip by a user. The housing includes a grip 26, a base 28, and a massage head 48. The housing components 26, 28, 48 are configured to withstand use under pressure required by massage. Injection molded plastic, for example, can be utilized for manufacture, with ABS plastic for relatively low cost and its strength, heat, and UL properties.

The massager 10 is designed to target all areas and muscles of the body. The massager 10 is shaped such that use by a single person on himself or herself can accommodate hard-to-reach areas. The massager 10 is designed to be of a weight reasonable for use by an individual without being cumbersome. Weight distribution is configured to favor the massage head 48 of the massager 10 to provide a pressure to the treated area.

The massager 10 is handheld and can be gripped by one or more hands. The massager 10 can provide therapeutic advantages, alleviate pain, stimulate circulation, relieve tension, provide flexibility to muscles and joints, and improve range of motion, and so forth. Additionally, the massager 10 can provide psychological benefits such as lessening depression and anxiety and increasing sleep quality. As such, the massager 10 combines multiple methods of therapy and multiple benefits into a single device, an all-in-one massager.

In one embodiment, the massager 10 can be utilized in massage parlors, physical therapy facilities, and the like, by a masseuse on a patient. In this embodiment, the massager 10 is manufactured in a durable, rugged manner, and may require two-handed use. This durable embodiment of the massager 10 is configured to be powered by an A/C power source.

In an alternative embodiment, the massager 10 can be utilized for home or personal use. This massager 10 is configured for one-handed use and can utilize a rechargeable battery, thereby being cordless.

The massager 10 includes a massage head capsule 30 within which a massage roller ball 42 rotates. The massage head capsule 30 is detachable from the massager head 48 at node 50 and is both replaceable and interchangeable, thereby providing the massager 10 with a plurality of massage options and a plurality of massage roller balls 42 of varying sizes. The massage head capsule 30 can rotate about the massage head 48 at node 50. The massage roller ball 42 is removable and replaceable with balls of varying sizes to accommodate different parts of the body of the user. The massage roller ball 42 is the primary element of the massager 10 to come in direct contact with the body of the user. The massage roller ball 42 is configured to be heated and cooled selectively and incrementally to provide variable hot and cold therapy. The massage roller ball 42 can be a metal, such as, for example, aluminum, or the like, such that it maintains adequate heat conductivity properties. The metal ball and massage fluid are chosen such that no adverse reaction occurs between the two with use of the massager 10. By way of example, the massage roller ball 42 can include a PTFE coating to protect against allergic reactions with a patient.

The massager 10 can further include a quick release button 18 disposed upon the massage head 48 of the massager 10 and configured to be actuated by the user to remove the massage capsule 30 from the massager 10 for interchangeability and replaceability.

The massager 10 can further include a removable protective cap 12 configured to protect the massage head capsule 30 and massage roller ball 42 from debris and damage. The protective cap 12 can be threaded.

The massager 10 also includes a thermoelectric assembly configured to selectively and incrementally heat and cool the massage roller ball 42. By way of example, and as depicted in the Figures, temperature band 46 serves this function and applies the desired temperature, hot or cold as variably selected, to the massage roller ball 42.

The temperature band 46 is generally conical in shape to house the massage roller ball 42 and allow it freedom of rotation while the massager 10 is in operation. A fractional portion of the surface of the massage roller ball 42 protrudes outwardly and resides outside of the massage head capsule 30 for contact with the body of the user. The temperature band 46 is provided in varying sizes to accommodate the varying sizes of the massage roller ball 42.

In various embodiments, the thermoelectric assembly, such as temperature band 46, configured to selectively and incrementally heat and cool the massage roller ball 42 can further includes one or more of a fan, a heat sink, a universal heat exchange device, and a conversion heat exchange device.

The massager 10 also includes a temperature control 16, such as a thermostat/thermometer, to control and measure a temperature of the thermoelectric device and massage roller ball 42. The temperature control 16 to control a temperature of the thermoelectric device is selectively and incrementally controlled by the user operating one or more of the plurality of user controls to heat and cool the massage roller ball 42. By way of example, the thermostat component can maintain and govern temperature of the massage roller ball 42 within a predetermined and restricted range, such as a low of 10-15 degrees Celsius and a high of 45-50 degrees Celsius. The thermometer component can maintain direct contact with the massage roller ball 42 and continuously record its temperature, the results of which can be shown in a temperature display window 38.

The massager 10 can also include a temperature display window 38 and a thermostat gauge 40 configured to provide the user with a current temperature of the massage roller ball 42. The thermostat gauge 40 can, for example, allow the user to select between low, medium, and high temperatures for hot and cold.

The massager 10 also includes a fluid reservoir 44 configured to hold a massage fluid for dispensing to the massage roller ball 42 and a fluid dispenser, such as on orifice at the top of the fluid reservoir 44, configured to control the release and volume of the massage fluid from the fluid reservoir 44. In at least one embodiment, the fluid reservoir 44 is a cartridge that is both interchangeable and disposable. By way of example, fluid reservoir 44 is a 3 ounce disposable thermoplastic container filled with massage fluid, or the like. The fluid reservoir 44 includes a cavity 64 at its base to allow the massage head capsule 30 to attach to the massage head 48.

The massager 10 can also include a turning wheel 60 having a stem disposed upon the massage head capsule 30 and configured for operative engagement to release massage fluid from the fluid reservoir 44. A small portion of the turning wheel 60 is exposed to allow manual rotation of the wheel, moving a disc to force the massage fluid out through the orifice to fill a saturating sponge 52. The turning wheel 60 is configured to be operable by a thumb of the user. The turning wheel 60 sits in thumb wheel cavity 62.

The massager 10 can further include a saturating sponge 52 disposed within the massage head capsule 30 and configured to absorb dispensed fluid and provide an even distribution of the fluid onto the massage roller ball 42 as it rotates over the saturating sponge 52. By way of example, the saturating sponge 52 is approximately 3 millimeters thick and fits flush inside the fluid reservoir 44 and over the orifice.

In operation, the massage head capsule 30 can be assembled for use by removing a seal, such as an aluminum sealing lid, from the fluid reservoir 44 and thread to the outer portion of the massage head capsule having the temperature band 46 and the massage roller ball 42. The massage roller ball 42 is now nested on the saturating sponge 52. A decoy, or empty, reservoir 44 can be utilized if the user does not desire to use massage fluid. The massage head capsule can be detached from the massage head for changing sizes, cleaning, adding massage fluid, and the like.

The massager 10 also includes a vibrator and rotator motor 54 configured to provide a vibration and rotation to the massager. The massager 10 is motor driven. As such, the massage head capsule 30 can both vibrate and rotate on protruding node 50. This pivoting movement of the massage head capsule 30 will produce a comfortable kneading effect to a desired area of treatment. The weight of the massage roller ball 42 provides deep penetrating. The massage roller ball 42 can be further configured to be magnetic to provide lasting and satisfactory results. In operation, the massage roller ball 42 evenly transfers massage fluid from the saturating sponge 52 to the treatment area on the body.

The massager 10 also includes a power source configured to provide power to the massager 10. The power source can include rechargeable battery 58 and a charger assembly. In this manner the massager is operable cordlessly. The charger assembly (not shown) is configured to recharge the rechargeable battery 58 and to provide power for use while rechargeable battery 58 is recharged. The massager 10 can also include at least one solar cell 14 to provide power. The solar cell 14 can also be configured to recharge the rechargeable battery 58. The massager 10 can be couple to a charging device, such as one with an A/C power source, through adapter cavity 32. A miniature LED lamp 36 can be utilized to illuminate green when the massager 10 is charged and ready for use. The miniature LED lamp 36 can be utilized to illuminate amber when the battery strength is too low to operate the massager 10.

The massager 10 also includes multiple user controls configured to variably control vibration, temperature, and power. By way of example, the user controls can include a vibration on/off switch 22, a power on/off switch 24, and a rotation on/off switch 20.

The massager 10 also includes a controller 54 configured to electronically couple and control the plurality of user controls, the power source, the fluid dispenser, the temperature control, and the thermoelectric assembly. The controller 54 can be a microcontroller. In the interest of safety, the controller 54 is further configured to automatically shut-off power to the massager 10 once a predetermined time has elapsed and once a predetermined temperature has been exceeded. This configuration provides a warning mechanism connected to the LED (light-emitting diode) 36 such that it can be illuminated when the warning event has occurred.

By way of example, when a particular temperature range has been exceeded, the warning mechanism actuates the LED to illuminate (red, for example) and for the massager 10 to beep and then automatically power off. Reset cavity 34 can be actuated to reset the red warning. By way of example, a pin, or the like, can be inserted into the reset cavity 34 to deactivate the red warning once the temperature band 46 and the massage roller ball 42 have returned to room temperature. The massager 10 should never be utilized when the temperature is out of range.

Although this technology has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the invention and are intended to be covered by the following claims.

What is claimed is:

1. A handheld massager comprising:
a housing dimensioned for handheld grip by a user;
a massage head capsule within which a massage roller ball rotates, wherein the massage roller ball is configured to be heated and cooled selectively and incrementally to provide variable hot and cold therapy;
a thermoelectric assembly configured to selectively and incrementally heat and cool the massage roller ball;
a temperature control to control a temperature of the thermoelectric device;
a fluid reservoir configured to hold a massage fluid for dispensing to the massage roller ball;
a fluid dispenser configured to control the release and volume of the massage fluid from the fluid reservoir;
a vibrator and rotator motor configured to provide a vibration and rotation to the massager;
a power source configured to provide power to the massager;
a plurality of user controls configured to variably control vibration, temperature, and power; and
a controller configured to electronically couple and control the plurality of user controls, the power source, the fluid dispenser, the temperature control, and the thermoelectric assembly.

2. The handheld massager of claim 1, wherein the massage head capsule is detachable from the massager housing and is both replaceable and interchangeable, thereby providing the massager with a plurality of massage options and a plurality of massage roller balls of varying sizes.

3. The handheld massager of claim 1, wherein the fluid dispenser is a cartridge that is both interchangeable and disposable.

4. The handheld massager of claim 1, wherein the thermoelectric assembly configured to selectively and incrementally heat and cool the massage roller ball further comprises a heat sink.

5. The handheld massager of claim 1, wherein the thermoelectric assembly configured to selectively and incrementally heat and cool the massage roller ball further comprises a fan.

6. The handheld thermal massager of claim 1, wherein the thermoelectric assembly configured to selectively and incrementally heat and cool the massage roller ball further comprises a universal heat exchange device.

7. The handheld massager of claim 1, wherein the thermoelectric assembly configured to selectively and incrementally heat and cool the massage roller ball further comprises a conversion heat exchange device.

8. The handheld massager of claim 1, wherein the massage roller ball is a metal ball.

9. The handheld massager of claim 1, wherein the power source further comprises a rechargeable battery and the handheld massager is configured to be cordless while in operation.

10. The handheld massager of claim 9, further comprising:
a charger assembly to recharge the rechargeable battery and to provide power for use while rechargeable battery is recharged.

11. The handheld massager of claim 1, wherein the power source further comprises a solar cell.

12. The handheld massager of claim 1, wherein the temperature control to control a temperature of the thermoelectric device is selectively and incrementally controlled by the user operating one or more of the plurality of user controls to heat and cool the massage roller ball.

13. The handheld massager of claim 1, wherein the plurality of user controls configured to variably control vibration, temperature, and power further comprises a vibration on/off switch and an intensity of vibration control.

14. The handheld massager of claim 1, wherein the controller is further configured to automatically shut-off power to the massager once a predetermined time has elapsed and once a predetermined temperature has been exceeded.

15. The handheld massager of claim 1, further comprising:
a saturating sponge disposed within the massage head capsule and configured to absorb dispensed fluid and provide an even distribution of the fluid onto the massage roller ball as it rotates over the saturating sponge.

16. The handheld massager of claim 1, further comprising:
a quick release button disposed upon the housing of the massager and configured to be actuated by the user to remove the massage capsule from the massager for interchangeability and replaceability.

17. The handheld massager of claim 1, further comprising:
a removable protective cap configured to protect the massage capsule and massage roller ball from debris and damage.

18. The handheld massager of claim 17, wherein the protective cap is threaded.

19. The handheld massager of claim 1, further comprising:
a temperature display window configured to provide the user with a current temperature of the massage roller ball.

20. The handheld massager of claim 1, further comprising:
a turning wheel having a stem disposed upon the massage capsule and configured for operative engagement to release massage fluid from the massage reservoir.
